(19) United States
(12) Patent Application Publication
(10) Pub. No.: US 2013/0190366 A1
(43) Pub. Date: Jul. 25, 2013

(54) SUBSTANTIALLY PURE SALTS OF FEBUXOSTAT AND PROCESSES FOR PREPARATION THEREOF

(71) Applicants: Shriprakash Dhar Dwivedi, Ahmedabad (IN); Ashok Prasad, Ahmedabad (IN); Rushikesh Udaykumar Roy, Ahmedabad (IN); Mayur Ramnikbhai Patel, Ahmedabad (IN)

(72) Inventors: Shriprakash Dhar Dwivedi, Ahmedabad (IN); Ashok Prasad, Ahmedabad (IN); Rushikesh Udaykumar Roy, Ahmedabad (IN); Mayur Ramnikbhai Patel, Ahmedabad (IN)

(73) Assignee: CADILA HEALTHCARE LIMITED, Ahmedabad, Gujarat (IN)

(21) Appl. No.: 13/579,418

(22) PCT Filed: Feb. 18, 2011
(86) PCT No.: PCT/IN11/00100

§ 371 Date: Oct. 18, 2012

(30) Foreign Application Priority Data
Feb. 19, 2010 (IN) 461/MUM/2010

(51) Int. Cl. C07D 277/56

(52) U.S. Cl. CPC C07D 277/56
USPC 514/365; 548/201

(57) ABSTRACT

Substantially pure salts of febuxostat of Formula (IA): wherein Y is Na⁺, K⁺, Li⁺, Mg²⁺, Ca²⁺, Zn²⁺, Ba²⁺, Sr²⁺, choline, epolamine and N⁺(R)₄ and processes for preparation thereof are disclosed.

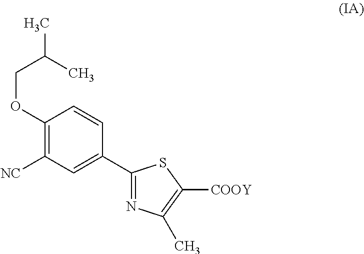

### FIELD OF THE INVENTION

The invention relates to processes for the preparation of substantially pure febuxostat. The invention also relates to the preparation of substantially pure salts of febuxostat. The invention also relates to pharmaceutical compositions that include the substantially pure febuxostat or salts thereof and use of said compositions for treating hyperuricemia.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Chemically, febuxostat is 2-(3-cyano-4-isobutyloxyphenyl)-4-methyl-5-thiazolecarboxylic acid having the structural Formula (I). It is indicated for the treatment of hyperuricemia.

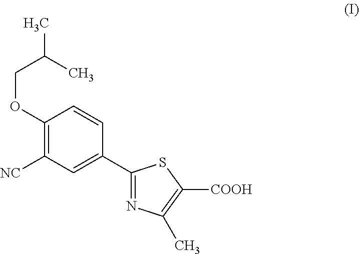 [SMILES: Cc1nc(-c2ccc(OCC(C)C)c(C#N)c2)sc1C(=O)O]

U.S. Pat. No. 5,614,520 discloses a process for the preparation of 2-arylthiazole derivative or pharmaceutically acceptable salts thereof, including febuxostat for treating diseases selected from consisting of gout or hyperuricemia and diseases associated with a production of interleukin. Febuxostat can be prepared as per the known procedures as described in Organic Reactions, Vol. 6, 367-409 (1951), or Heterocyclic Compounds, Vol. 34 (1978). The arylthiazoles can be prepared by the process as disclosed in scheme-1.

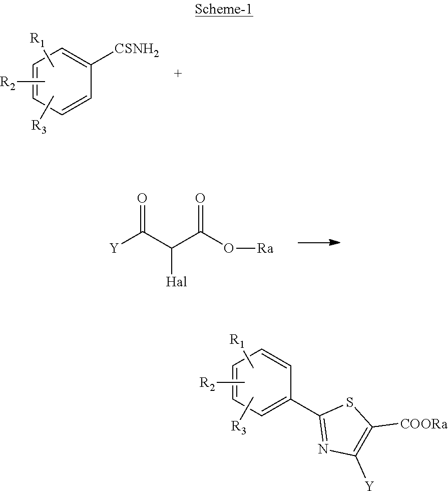

Hal represents a halogen atom, and Ra represents a C1-4 alkyl group. R₁, R₂ and R₃ are defined in the specification.

Japanese Patent No. 06345724 discloses a method for the preparation of 2-(3-cyanophenyl)thiazole derivative. The title compound or a pharmacologically acceptable salt thereof is prepared by reacting 4-nitrobenzonitrile and KCN at 100° C. in DMSO and iso-Butyl bromide and K₂CO₃ were added followed by stirring the mixture at 80° C. for 8 h to give intermediate with 50% yield. The latter compound was stirred with thioacetamide in 6 N HCl in DMF at 45° C. and additional HCl/DMF and thioacetamide were added followed by stirring the mixture for 24 h to give arylthioacetamide derivative in 92% yield. A solution of the latter compound and ethyl chloro-acetoacetate in EtOH was heated with stirring at 100° C. for 100 min to give febuxostat.

Heterocycles Vol. 47, No. 2 (1998) p 857 discloses a process for the preparation of febuxostat (I) by using 4-isobutoxy-1,3-benzenedicarbonitrile.

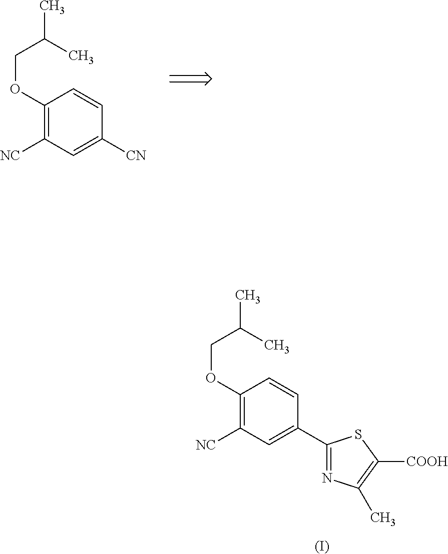 [SMILES: CC(C)COc1ccc(C#N)cc1C#N ; Cc1nc(-c2ccc(OCC(C)C)c(C#N)c2)sc1C(=O)O ; "(I)"]

The process parameters provided in Heterocycles discloses introduction of cyano group to 4-nitrobenzonitrile and converting it to 4-alkoxy-1,3-benzenedicarbonitrile in a one-pot process. Further the obtained 4-alkoxy-1,3-benzenedicarbonitrile can be converted to febuxostat (I) as shown in reaction scheme-2.

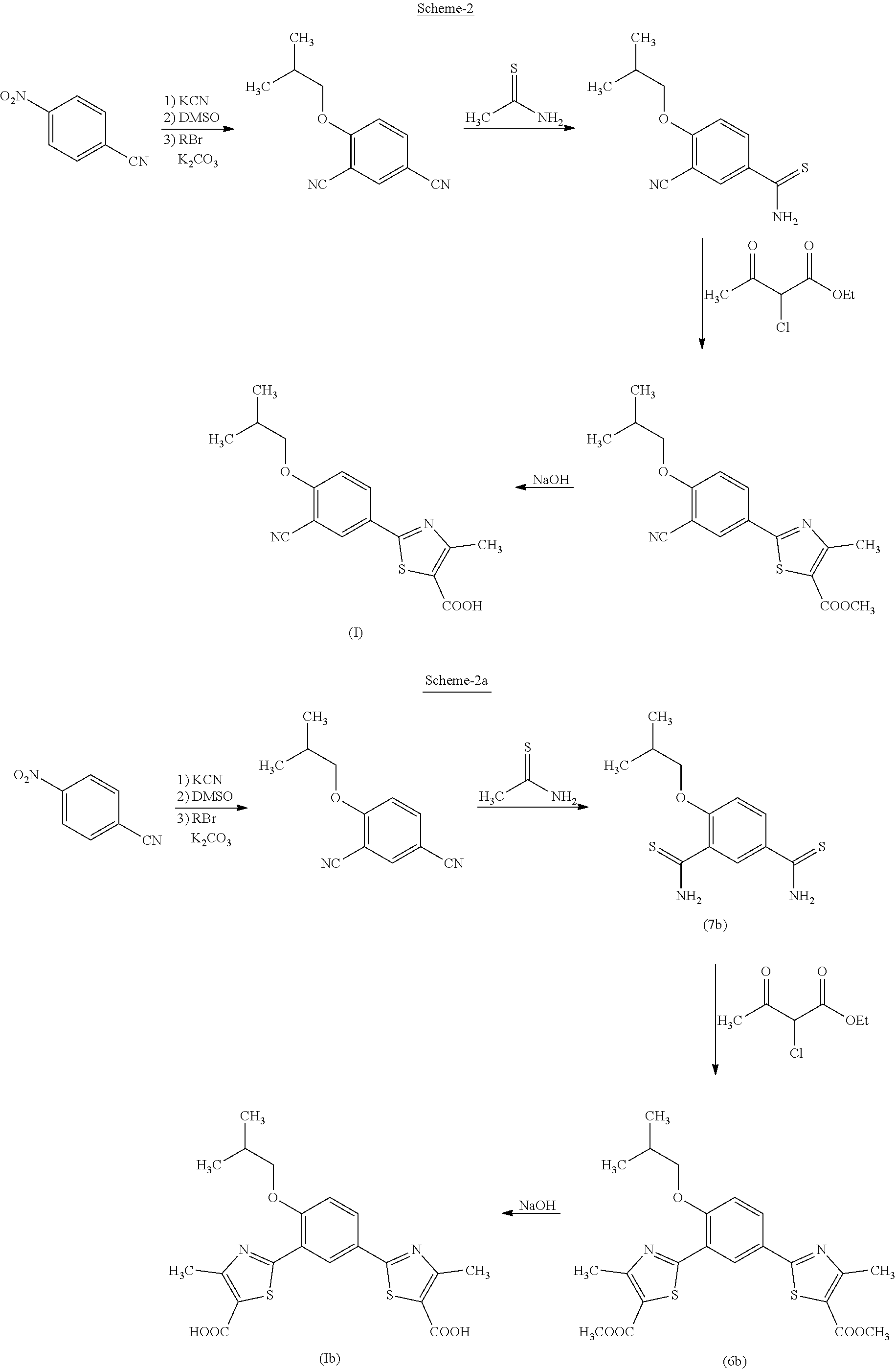

The compound 4-alkoxy-1,3-benzenedicarbonitrile undergoes side reaction by the formation of byproduct of dimer impurity of formula (7b). The entire reaction pathway proceeds similar to the formation of febuxostat (I) as shown above in the scheme-2a. Thus, the process provided in the prior art is not suitable for the formation of febuxostat (I) in high yield and purity.

U.S. Pat. No. 6,225,474 B1 discloses novel crystalline forms of febuxostat viz. crystals A, B, C, D, and G and an amorphous form and a method for producing them. The specification, in column-7, line-25 onwards, preparation of crystalline Form has been provided by using sodium salt of febuxostat.

Solubility and Stability are important characteristic of a salt form that can affect its suitability for use as a drug. Where aqueous solubility is low, i.e. less than 10 mg/ml, the dissolution rate at in vivo administration can be rate limiting in the absorption process leading to poor bioavailability. Therefore, poorly soluble drug substance requires special efforts in formulation development to achieve desired results. The stability of drug product is important parameter for its pharmaceutical use.

Although febuxostat provides good pharmaceutical activity, it would be beneficial to find other forms of febuxostat; in particular, febuxostat salts having advantageous properties for pharmaceutical use.

The method reported in the prior art doesn't provide any useful method for the preparation of febuxostat with high purity. Thus, there are still requirements on improved process in the art for obtaining febuxostat with high purity. Hence, the inventors of the present invention provides improved processes for the preparation of febuxostat in high purity by preparing its alkali metal salts in their crystalline forms and converting alkali metal salts of febuxostat to febuxostat (I).

### SUMMARY OF THE INVENTION

In one general aspect there are provided substantially pure salts of febuxostat of Formula (IA),

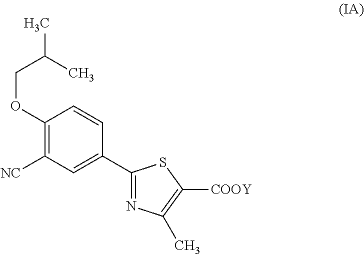

wherein Y is Na⁺, K⁺, Li⁺, Mg²⁺, Ca²⁺, Zn²⁺, Ba²⁺, Sr²⁺, choline, epolamine and N⁺(R)₄
R is alkyl with 1-4 carbon atoms. The process includes:
(a) treating ethyl 2-(3-cyano-4-isobutoxyphenyl)-4-methylthiazole-5-carboxylate with a base;
(b) heating till the conversion of febuxostat free acid to salt; and
(c) isolating the febuxostat of Formula (IA).

The process may produce the substantially pure salts of febuxostat having purity greater than 99% by area percentage of HPLC. In particular, it may produce the pure salts of febuxostat having purity greater than 99.5% by area percentage of HPLC.

The process may produce the substantially pure salts of febuxostat which are substantially free from dimer impurities of Formula (7b), (6b) and (1b).

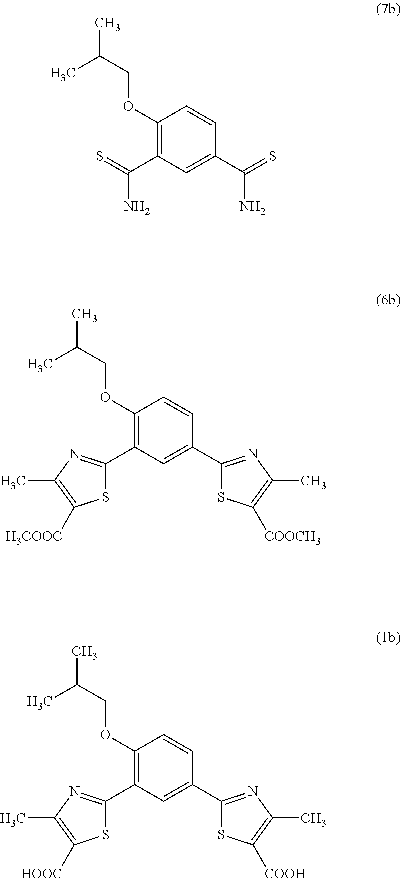

In another general aspect there is provided febuxostat sodium of Formula (IA-a),

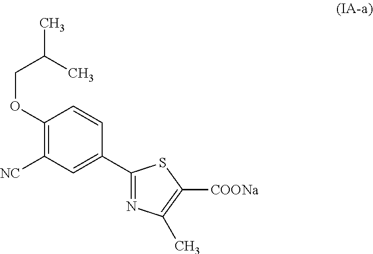

characterized by at least one of the following:
(a) x-ray diffraction pattern having characteristic peaks at 5.5°, 11.2° and 16.8°±0.2° 2θ; or
(b) Infrared spectroscopic values at about 2910, 2227, 1170, 1130 and 1016 cm⁻¹; or
(c) water content from about 3% to about 6%.

In another general aspect there is provided febuxostat sodium of Formula (IA-a) having a purity greater than 99% by area percentage of HPLC.

In one general aspect there is provided febuxostat potassium of Formula (IA-b),

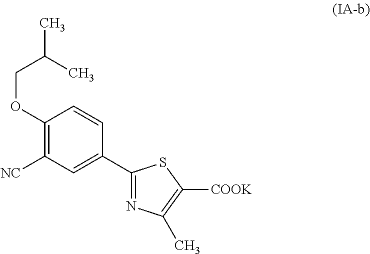

characterized by at least one of the following:
(a) x-ray diffraction pattern having characteristic peaks at 7.8°, 10.7° and 24.7°±0.2° 2θ; or
(b) Infrared spectroscopic values at about 3404, 2962, 2225, 1604, 1512, 1132 and 1014 cm⁻¹; or
(c) water content from about 5% to about 8%.

In another general aspect there is provided febuxostat potassium of Formula (IA-b) having purity greater than 99% by area percentage of HPLC.

In another general aspect there is provided a process for the preparation of substantially pure febuxostat. The process includes obtaining a solution or a suspension of febuxostat in water having alkaline pH; adding one or more ester solvents; acidifying with acid; heating reaction mixture; separating organic phase; and isolating the substantially pure febuxostat.

In another general aspect there is provided a process for the preparation of substantially pure febuxostat. The process includes:
(a) treating febuxostat with a suitable base to obtain febuxostat salt of Formula (IA)

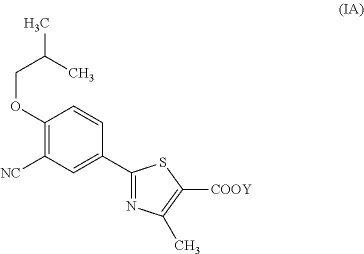

wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms;
(b) treating the febuxostat salt of Formula (IA) with an acid; and
(c) isolating the substantially pure febuxostat of Formula (I).

The process may produce the substantially pure febuxostat having purity greater than 99% by area percentage of HPLC. In particular, it may produce the pure febuxostat having a purity greater than 99.5% by area percentage of HPLC. The process may produce the substantially pure febuxostat which is substantially free from dimer impurities of Formula (7b), (6b) and (1b).

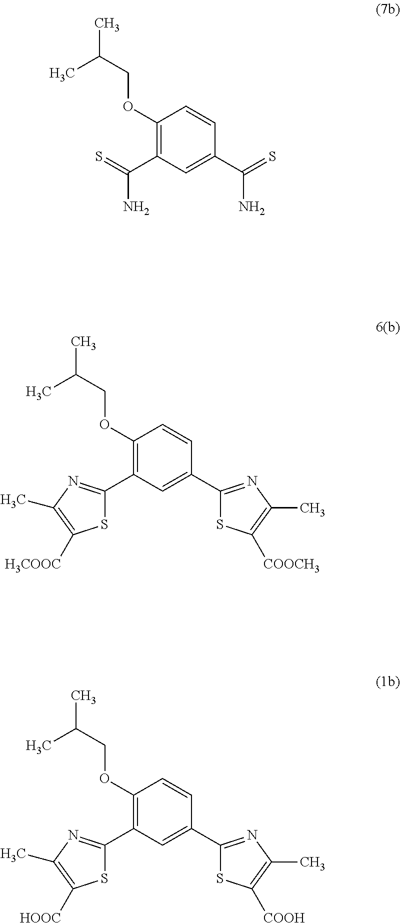

In another general aspect there are provided pharmaceutical compositions that include substantially pure febuxostat or substantially pure salts of febuxostat, and one or more pharmaceutically acceptable carriers, excipients or diluents.

The details of one or more embodiments of the invention are set forth in the description below. Other features, objects and advantages of the invention will be apparent from the description and claims.

### BRIEF DESCRIPTION OF DRAWINGS

FIG. 1: X-ray powder diffraction pattern of crystalline febuxostat prepared as per Example-2.

FIG. 2: X-ray powder diffraction pattern of crystalline febuxostat sodium prepared in Example-1.

FIG. 3: X-ray powder diffraction pattern of crystalline febuxostat potassium prepared in Example-4.

FIG. 4: IR spectrum of febuxostat sodium prepared in Example-1.

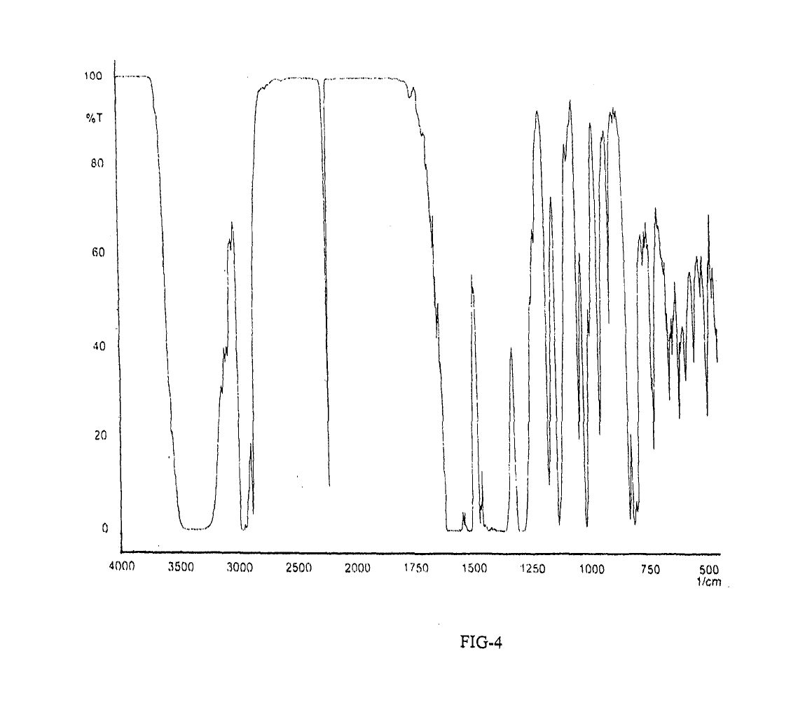

FIG. 5: IR spectrum of febuxostat potassium prepared in Example-4.

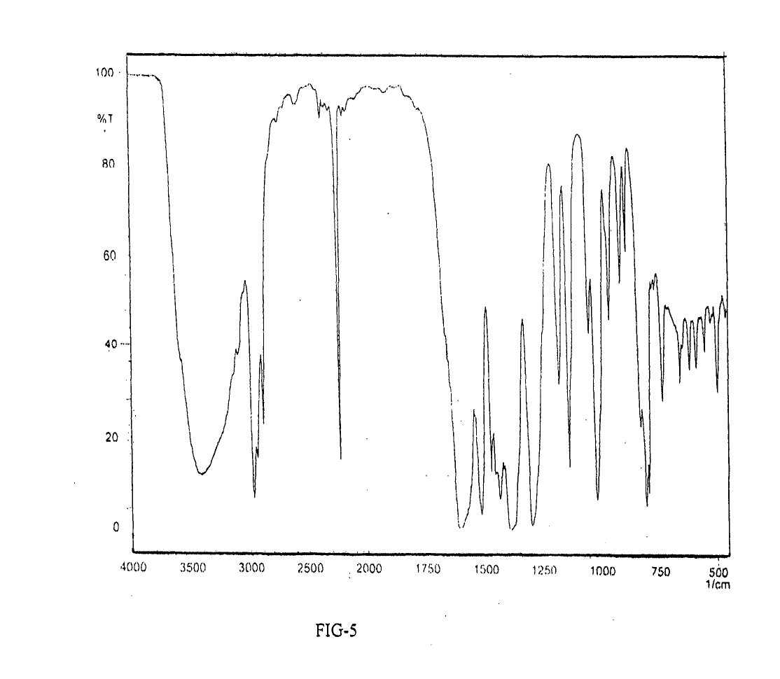

### DETAILED DESCRIPTION OF THE INVENTION

The present inventors have found that by preparing substantially pure salts of febuxostat (IA), one can eliminate or minimize the formation of dimer impurity as shown in scheme-2a to very low levels. Thus, febuxostat salts of Formula (IA) can be converted to febuxostat (I) with high purity.

As used herein the term “isolation” includes filtration, filtration under vacuum, centrifugation, and decantation. The product obtained may be further or additionally dried to achieve the desired moisture values. For example, the product may be dried in a tray drier, dried under vacuum and/or in a Fluid Bed Drier.

Optionally, the solution, prior to any solids formation, can be filtered to remove any undissolved solids, solid impurities and the like prior to removal of the solvent. Any filtration system and filtration techniques known in the art can be used.

As used herein, the terms “triturating”, “slurrying” and “suspending” are interchangeable, and refer to a process carried out in a heterogeneous mixture where complete dissolution does not occur. Also, heating the suspension or slurry can result in a homogenous mixture where complete or partial dissolution occurs at an elevated temperature or ambient temperature.

“Suitable solvent” means a single or a combination of two or more solvents.

“Substantially pure” means febuxostat (I) prepared by the process of the present invention is substantially free from dimer impurities of Formula (7b), (6b) and (1b) respectively. The impurities (7b), (6b) and (1b) individually are less than about 0.5% by area percentage of HPLC; In particular it is less than about 0.25%. Most particularly, it is less than about 0.1% by area percentage of HPLC.

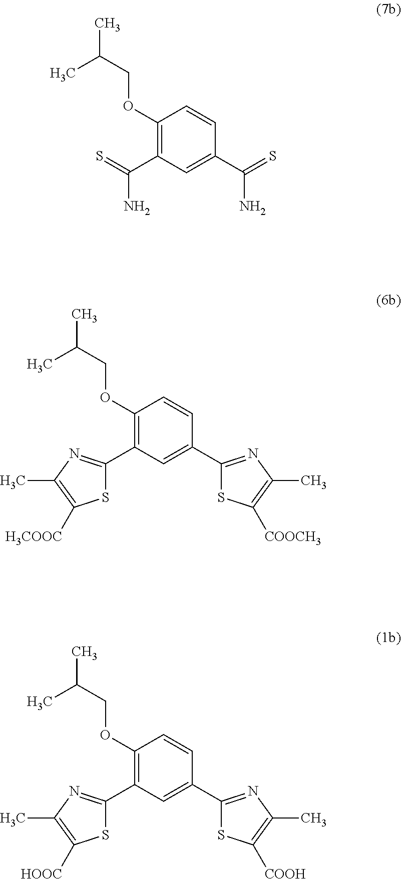

Embodiments of the process may include one or more of the following features. For example, the solution or suspension may be obtained by dissolving or suspending febuxostat in a suitable solvent. Alternatively, such a solution may be obtained directly from a reaction mixture in a process in which febuxostat is formed. The solvent containing febuxostat may be heated to obtain a solution. It can be heated from about 30° C. to about boiling point of the solvent used, for example from about 30° C. to about 100° C.

The term “obtaining” includes dissolving, slurrying, stirring or a combination thereof.

In one aspect there are provided substantially pure salts of febuxostat of Formula (IA),

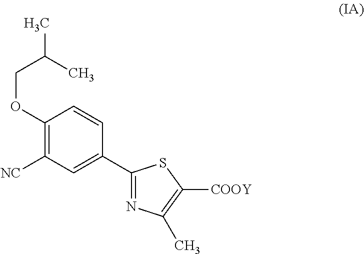

wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms.

In particular, the invention provides substantially pure salts of febuxostat of Formula (IA) in solid isolated form;

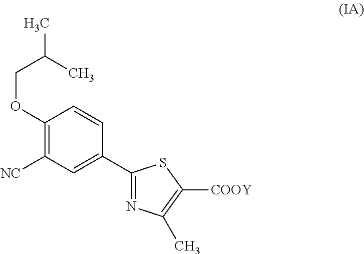

wherein Y is Na⁺, K⁺, Li⁺, Mg²⁺, Ca²⁺, Zn²⁺, Ba²⁺, Sr²⁺, choline, epolamine and N⁺(R)₄
R is alkyl with 1-4 carbon atoms.

In another aspect there is provided a crystalline febuxostat sodium of Formula (IA-a),

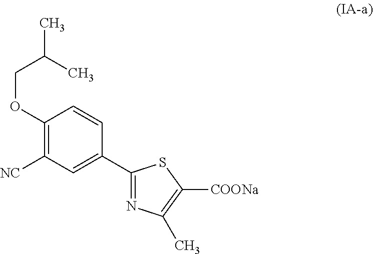

characterized by atleast one of the following:
(a) x-ray diffraction pattern having characteristic peaks at 5.5°, 11.2° and 16.8°±0.2° 2θ; or
(b) Infrared spectroscopic values at about 2910, 2227, 1170, 1130 and 1016 cm⁻¹; or
(c) having from 3 to 6% of water content.

In another aspect, there is provided febuxostat sodium of Formula (IA-a) is having purity greater than 99.5% by area percentage of HPLC.

According to another aspect, there is provided a crystalline febuxostat potassium of Formula (IA-b),

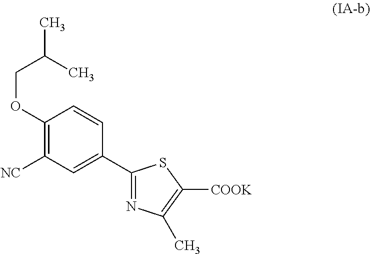

characterized by atleast one of the following:
(a) x-ray diffraction pattern having characteristic peaks at 7.8°, 10.7° and 24.7°±0.2° 2θ; or
(b) Infrared spectroscopic values at 3404, 2962, 2225, 1604, 1512, 1132 and 1014 cm⁻¹; or
(c) having from 5 to 8% of water content.

According to further aspect, there is provided febuxostat potassium of Formula (IA-b) having purity by purity greater than 99.5% by area percentage of HPLC.

According to yet another aspect, there is provided a process for the preparation of febuxostat salts of Formula (IA)

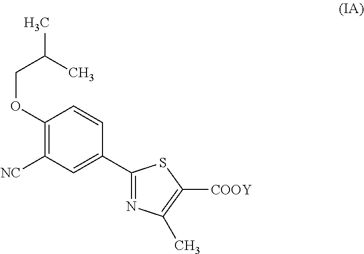

wherein Y is Na⁺, K⁺, Li⁺, Mg²⁺, Ca²⁺, Zn²⁺, Ba²⁺, Sr²⁺, choline, epolamine and N⁺(R)₄
R is alkyl with 1-4 carbon atoms,
the process comprising:
(a) hydrolyzing ethyl 2-(3-cyano-4-isobutoxyphenyl)-4-methylthiazole-5-carboxylate with a base in suitable organic solvent;
(b) heating; and
(c) isolating the febuxostat salts of Formula (IA).

In particular, the substantially pure salts of febuxostat of the present invention include sodium, potassium, magnesium, calcium, lithium, zinc, barium, strontium, choline, epolamine, t-butyl amine, n-butyl amine, ethylamine, ammonia etc.

In general procedure, the process for the preparation of febuxostat (I) may include reacting ethyl 2-(3-cyano-4-isobutoxyphenyl)-4-methylthiazole-5-carboxylate with base selected from one or more of sodium hydroxide, potassium hydroxide, lithium hydroxide, calcium hydroxide, barium hydroxide, strontium hydroxide, zinc hydroxide, choline hydroxide, epolamine hydroxide, sodium carbonate, potassium carbonate, lithium carbonate, magnesium carbonate, calcium carbonate, sodium bicarbonate, potassium bicarbonate, lithium bicarbonate, sodium methoxide, potassium t-butoxide, magnesium methoxide and the like. In particular, the base may be sodium hydroxide or potassium hydroxide.

The hydrolysis of ethyl 2-(3-cyano-4-isobutoxyphenyl)-4-methylthiazole-5-carboxylate can be performed in an organic solvent selected from aliphatic alcohols like methanol (MeOH), ethanol (EtOH), n-propanol, isopropanol (IPA), n-butanol, tert-amyl alcohol (t-AmOH); aliphatic ketones like acetone, methylethylketone (MEK), methylisobutyl ketone (MIBK), polar aprotic solvents like dimethylformamide (DMF), dimethylacetamide (DMAc), dimethylsulfoxide (DMSO), N-methylpyrrolidone (NMP) and the like. In particular, the suitable organic solvent is isopropanol (IPA) to obtain febuxostat salts of Formula (IA).

Embodiments of the process involve the formation of solution or suspension by heating the reaction mixture in a suitable solvent system. The heating of the reaction mixture can be at about the boiling point of the solvent system, specifically from about 25° C. to about 100° C. In particular, it may be heated from about 50° C. to about 80° C. More particular, it may be heated from about 60° C. to about 80° C.

The product obtained can be isolated by one or more of filtration, filtration under vacuum, evaporation, decantation, centrifugation, drying and drying under vacuum. The product can also be isolated upon cooling at an ambient temperature. The isolated product can optionally be washed with IPA before drying.

In yet another aspect, there is provided a process for preparation of substantially pure febuxostat, the process comprising:
(a) obtaining a solution or a suspension containing febuxostat in water having alkaline pH;
(b) adding one or more ester solvents;
(c) acidifying with acid;
(d) heating the reaction mixture;
(e) separating an organic phase; and
(f) isolating the substantially pure febuxostat.

In general, the febuxostat in solution or suspension form in alkaline pH is in the form of febuxostat alkali metal salt. The ester solvent in step (b) is one or more of ethyl acetate, n-propyl acetate, isopropyl acetate, n-butyl acetate, tertbutyl acetate and the like.

Embodiments of the process includes acidification of reaction mixture. Suitable acids are one or more of acetic acid, formic acid, hydrochloric acid, sulfuric acid, phosphoric acid and the like.

In general, the heating of reaction mixture is performed from about ambient temperature to about reflux temperature. In particular, heating can be performed at 35° C. to about 60° C. The product obtained can be isolated by one or more of filtration, filtration under vacuum, evaporation, decantation, centrifugation, drying and drying under vacuum. In particular, the product is isolated by removal of organic solvent by evaporation i.e. distillation. The remaining solution after solvent evaporation is cooled to ambient temperature of less than 25° C. to precipitate substantially pure febuxostat (I). The substantially pure febuxostat (I) is dried.

Febuxostat (I) thus obtained by the process as discussed above can additionally be purified with a suitable organic solvent to obtain substantially pure febuxostat (I). The suitable organic solvents selected for the purification of febuxostat are one or more of methanol, ethanol, n-propanol, isopropanol, n-butanol, tert-amyl alcohol; aliphatic ketones like acetone, methylethylketone, methylisobutyl ketone; esters like ethyl acetate, n-butyl acetate, tert-butyl acetate, acetonitrile, DMF, DMAc, N-methylpyrrolidone, cyclohexane, n-heptane, n-hexane, methylene dichloride, and the like, and mixture thereof with water.

The substantially pure salts of febuxostat are typically in a crystalline form. Crystalline forms include febuxostat salts with alkali metal or alkaline earth metal of suitable bases as described above, anhydrates, hydrates, and solvates. The febuxostat salts (IA) can be isolated, if desired, by precipitation, evaporation, spray drying, or other conventional techniques known in the art.

In one of the aspect of the invention, there is provided febuxostat substantially free from dimer impurities of Formula (7b), (6b) and (1b).

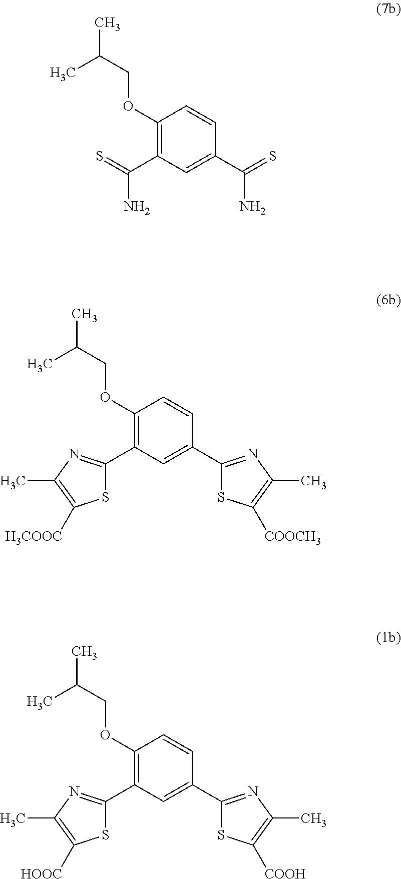

In still another aspect of the invention, there is provided substantially pure salts of febuxostat of Formula (IA),

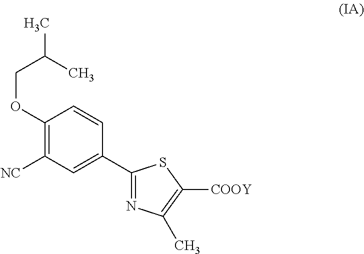

wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms, which are substantially free from dimer impurities of Formula (7b), (6b) and (1b).

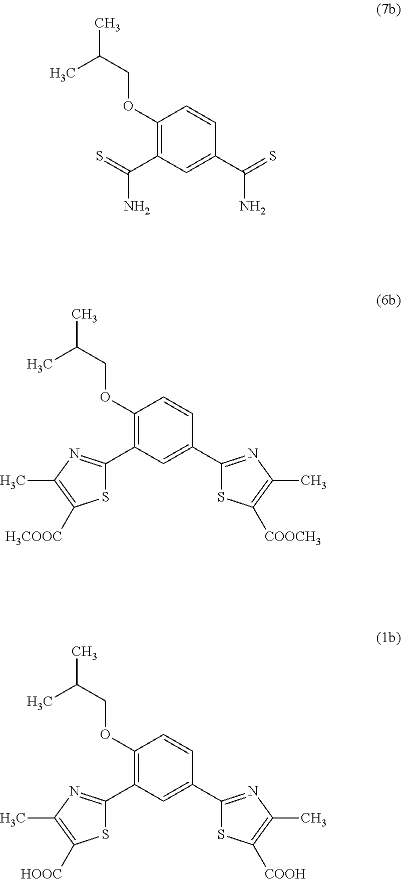

According to one of the aspect, the present invention provides a process for the preparation of substantially pure febuxostat of Formula (I)

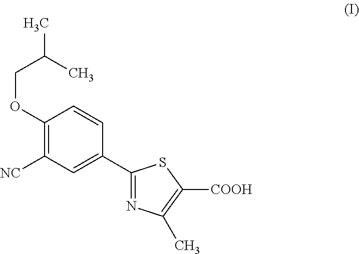 [SMILES: Cc1nc(-c2ccc(OCC(C)C)c(C#N)c2)sc1C(=O)O]

the process comprising:
(a) treating febuxostat with a suitable base to obtain febuxostat salt of Formula (IA)

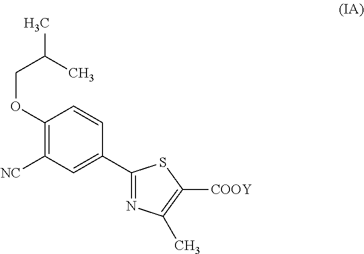

wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms;
(b) treating the febuxostat salt of Formula (IA) with an acid; and
(c) isolating the substantially pure febuxostat of Formula (I).

In general, the suitable base can be selected from one or more of sodium hydroxide, potassium hydroxide, lithium hydroxide, calcium hydroxide, barium hydroxide, strontium hydroxide, zinc hydroxide, choline hydroxide, epolamine hydroxide, sodium carbonate, potassium carbonate, lithium carbonate, magnesium carbonate, calcium carbonate, sodium bicarbonate, potassium bicarbonate, lithium bicarbonate, sodium methoxide, potassium t-butoxide, magnesium methoxide, and the like.

The embodiments of the process includes treating the febuxostat salt of Formula (IA) with an acid selected from one or more of acetic acid, formic acid, hydrochloric acid, sulfuric acid, phosphoric acid, and the like.

According to the preferred aspect, the present invention relates to the use of the compound of Formula (IA) in the process of manufacturing of 2-(3-cyano-4-isobutoxyphenyl)-4-methylthiazole-5-carboxylic acid.

Further aspect of the present invention related to a pharmaceutical composition comprising febuxostat salt of Formula (IA),

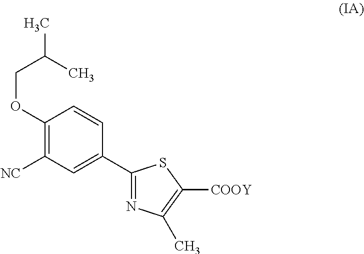

wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms, and one or more pharmaceutically acceptable carriers, excipients or diluents.

The process for the preparation of ethyl 2-(3-cyano-4-isobutoxyphenyl)-4-methylthiazole-5-carboxylate can be depicted by the reaction pathway which is reported in the art and disclosed herein as reference in its entirety.

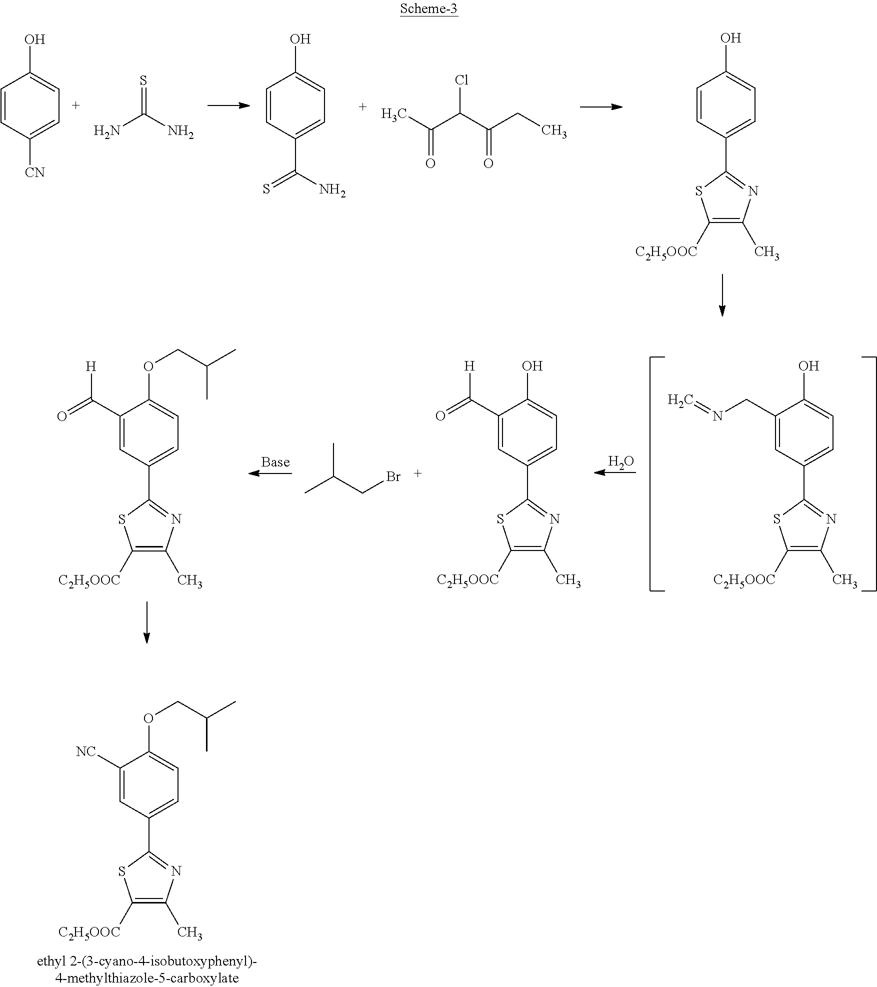

The invention also encompasses pharmaceutical compositions comprising Febuxostat salt (IA) of the invention. As used herein, the term “pharmaceutical compositions” or “pharmaceutical formulations” includes tablets, pills, powders, liquids, suspensions, emulsions, granules, capsules, suppositories, or injection preparations.

Pharmaceutical compositions containing the Febuxostat salts of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

Any excipient commonly known and used widely in the art can be used in the pharmaceutical composition.

Carriers used include, but are not limited to, lactose, white sugar, sodium chloride, glucose, urea, starch, calcium carbonate, kaolin, crystalline cellulose, salicylic acid, and the like. Binders used include, but are not limited to, water, ethanol, propanol; simple syrup, glucose solutions, starch solutions, gelatin solutions, carboxymethyl cellulose, shellac, methyl cellulose, potassium phosphate, polyvinyl pyrrolidone, and the like.

Disintegrating agents used include, but are not limited to, dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, and the like.

Disintegration inhibitors used include, but are not limited to, white sugar, stearin, coconut butter, hydrogenated oils, and the like. Absorption accelerators used include, but are not limited to, quaternary ammonium base, sodium laurylsulfate, and the like.

Wetting agents used include, but are not limited to, glycerin, starch, and the like. Adsorbing agents used include, but are not limited to, starch, lactose, kaolin, bentonite, colloidal salicylic acid, and the like. Lubricants used include, but are not limited to, purified talc, stearates, boric acid powder, polyethylene glycol, and the like.

Tablets can be further coated with commonly known coating materials such as sugar coated tablets, gelatin film coated tablets, tablets coated with enteric coatings, tablets coated with films, double layered tablets, and multi-layered tablets.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, and semi-synthesized glycerides.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic.

Additional ingredients, such as dissolving agents, buffer agents, and analgesic agents may be added. If necessary, coloring agents, preservatives, perfumes, seasoning agents, sweetening agents, and other medicines may also be added to the desired preparations.

The amount of febuxostat salt contained in a pharmaceutical composition for treating hyperuricemia and gout should be sufficient to treat, ameliorate, or reduce the symptoms associated with hyperuricemia and gout. Preferably, Rosuvastatin is present in an amount of about 1% to about 60% by weight, and more preferably from about 1% to about 35% by weight of the dose.

The pharmaceutical compositions of the invention may be administered in a variety of methods depending on the age; sex, and symptoms of the patient. For example, tablets, pills, solutions, suspensions, emulsions, granules and capsules may be orally administered. Injection preparations may be administered individually or mixed with, injection transfusions such as glucose solutions and amino acid solutions intravenously.

If necessary, the injection preparations may be administered intramuscularly, intracutaneously, subcutaneously. Suppositories may be administered into the rectum. The dosage of a pharmaceutical composition for treating hyperuricemia and gout according to the invention will depend on the method of use, the age, sex, and condition of the patient.

The novel salts and process for its preparation described in the present invention is demonstrated in examples illustrated below. These examples are provided as illustration only and therefore should not be construed as limitation of the scope of invention.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

### Preparation of Febuxostat Sodium (IA-a)

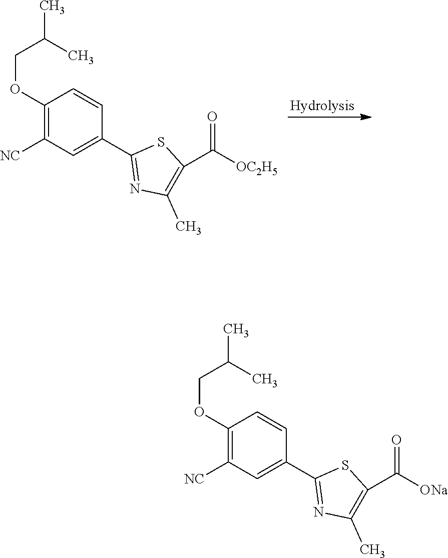

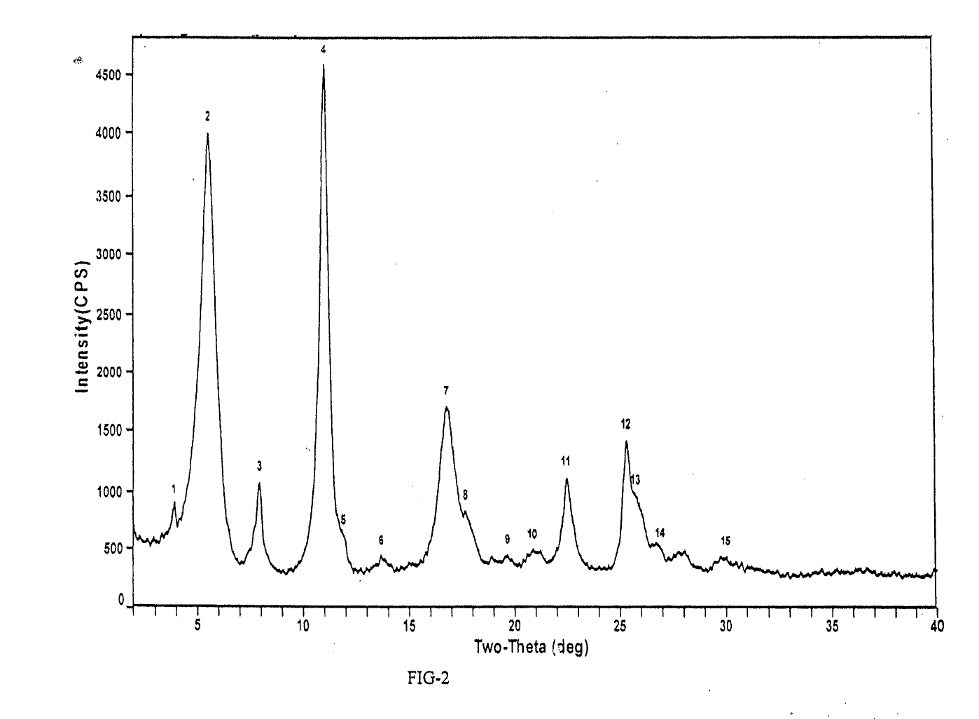

100 g of ethyl 2-(3-cyano-4-isobutoxyphenyl)-4-methylthiazole-5-carboxylate, 500 mL isopropanol and 128 g sodium hydroxide solution in water were taken in a round bottom flask at 35° C. The reaction mixture was heated at 75° C. for 30 minutes. After the completion of the reaction as monitored by TLC, the reaction mixture was gradually cooled to 30° C. and stirred for 2 hours. The reaction mixture was filtered and washed with isopropanol. The product was dried in hot air oven at about 65° C. to 70° C. to obtain 79% febuxostat sodium. HPLC Purity >99.5%. The XRD of febuxostat sodium is attached as FIG. 2.

### Example 2

### Preparation of Febuxostat (I)

50 g of febuxostat sodium, 500 mL water and 125 mL ethyl acetate at 25° C. were stirred for 15 minutes at room temperature. The reaction mixture was acidified with 2.5% HCl solution and heated to 50° C. The reaction mixture was stirred for 30 minutes and organic layer was separated. The organic layer was washed with water. The ethyl acetate was distilled partially and cooled to 10° C. The product was filtered and washed with ethyl acetate (500 mL) and dried in vacuum dryer oven at 65° C. to 70° C. to obtain 84% pure febuxostat. HPLC Purity >99.5%.

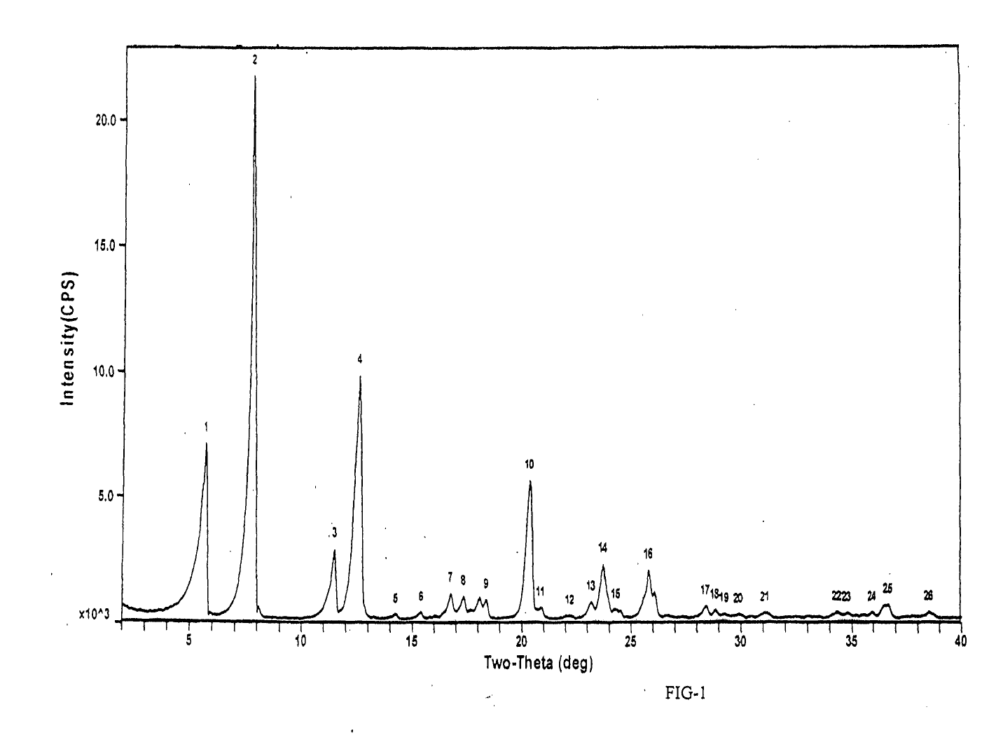

The XRPD of febuxostat is attached as FIG. 1.

### Example 3

### Preparation of Febuxostat Sodium (IA-a)

3.2 N sodium hydroxide solution was added to a reaction mass containing (50.0 g) 2-(4-isobutoxy-3-cyanophenyl)-4-methylthiazole-5-ethyl carboxylate in (250.0 ml) isopropyl alcohol. The reaction mixture was stirred and heated to get complete sodium salt of febuxostat. The febuxostat sodium was isolated by filtration. The moisture content of the said compound is 3.49%. HPLC purity: >99%

The XRPD of febuxostat sodium is attached as FIG. 2.

### Example-4

### Preparation of Febuxostat Potassium (IA-b)

3.7 N potassium hydroxide solution was added to a reaction mass of (50.0 g) 2-(4-isobutoxy-3-cyanophenyl)-4-methylthiazole-5-ethyl carboxylate in (250.0 ml) isopropyl alcohol. The reaction mixture was stirred and heated to get complete potassium salt of febuxostat. The febuxostat potassium was isolated by filtration. The moisture content of the said compound is 6.03%. HPLC purity: >99%

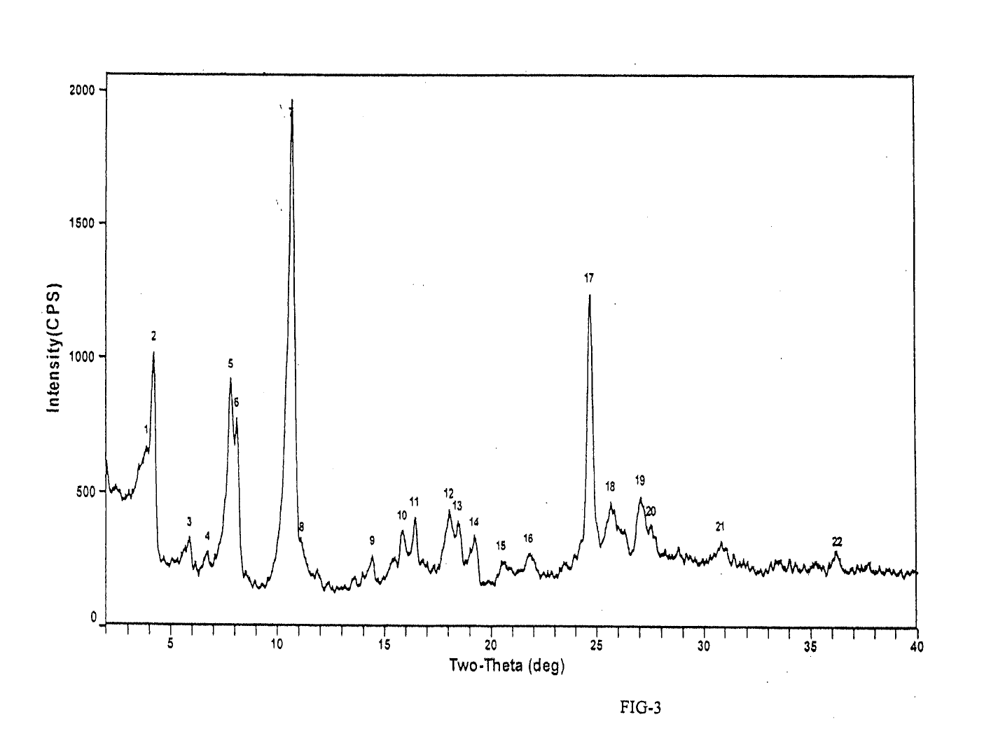

The XRPD of potassium salt of febuxostat is attached as FIG. 3.

### Example-5

### Preparation of Febuxostat (I)

3.2 N sodium hydroxide solution was added to a reaction mass containing (50.0 g) 2-(4-isobutoxy-3-cyanophenyl)-4-methylthiazole-5-ethyl carboxylate in (250.0 ml) isopropyl alcohol. The reaction mixture was stirred and heated and then acidified with acetic acid to get the febuxostat. HPLC purity: >99.5%.

While the invention has been described in terms of its specific embodiments, certain modifications and equivalents will be apparent to those skilled in the art and are intended to be included within the scope of the invention.

We claim:

1. Substantially pure salts of febuxostat of Formula (IA), 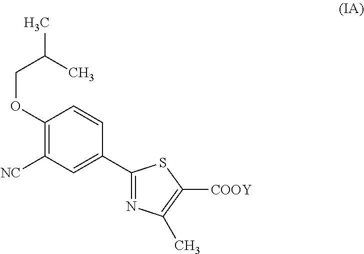
wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms.

2. Febuxostat sodium of Formula (IA-a), 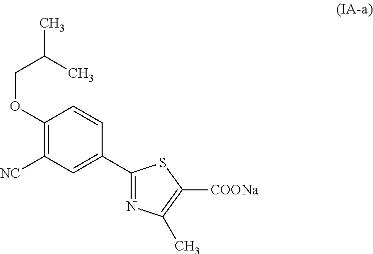
characterized by at least one of the following:
(a) x-ray diffraction pattern having characteristic peaks at 5.5°, 11.2° and 16.8°±0.2° 2θ; or
(b) infrared spectroscopic values at about 2910, 2227, 1170, 1130 and 1016 cm⁻¹; or
(c) water content from about 3% to about 6%.

3. Febuxostat sodium having purity greater than 99.5% by area percentage of HPLC.

4. Febuxostat potassium of Formula (IA-b), 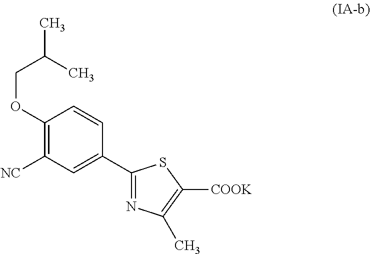
characterized by at least one of the following:
(a) x-ray diffraction pattern having characteristic peaks at 7.8°, 10.7° and 24.7° 0.2° 2θ; or
(b) infrared spectroscopic values at about 3404, 2962, 2225, 1604, 1512, 1132 and 1014 cm⁻¹; or
(c) water content from about 5% to about 8%.

5. Febuxostat potassium having purity greater than 99.5% by area percentage of HPLC.

6. A process for the preparation of febuxostat salts of Formula (IA) 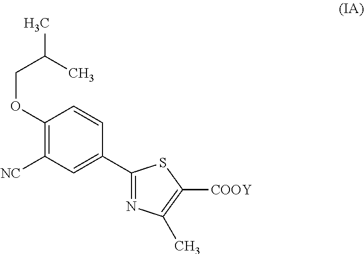
wherein Y is Na⁺, K⁺, Li⁺, Mg²⁺, Ca²⁺, Zn²⁺, Ba²⁺, Sr²⁺, choline, epolamine and N⁺(R)₄, wherein
R is alkyl with 1-4 carbon atoms,
the process comprising:
(a) hydrolyzing ethyl 2-(3-cyano-4-isobutoxyphenyl)-4-methylthiazole-5-carboxylate with a base in one or more organic solvents;
(b) heating; and
(c) isolating the febuxostat salts of Formula (IA).

7. The process as claimed in claim 6, wherein the base comprises one or more of sodium hydroxide, potassium hydroxide, lithium hydroxide, calcium hydroxide, barium hydroxide, strontium hydroxide, zinc hydroxide, choline hydroxide, epolamine hydroxide, sodium carbonate, potassium carbonate, lithium carbonate, magnesium carbonate, calcium carbonate, sodium bicarbonate, potassium bicarbonate, lithium bicarbonate, sodium methoxide, potassium t-butoxide, magnesium methoxide, and the like.

8. The process as claimed in claim 6, wherein the organic solvent comprises one or more of aliphatic alcohols, aliphatic ketones, polar aprotic solvents, or mixtures thereof.

9. The process as claimed in claim 8, wherein the alcohol comprises one or more of methanol, ethanol, n-propanol, isopropanol, n-butanol, and tert-amyl alcohol.

10. The process as claimed in claim 8, wherein the aliphatic ketones comprises one or more of acetone, methylethylketone, and methylisobutyl ketone.

11. The process as claimed in claim 8, wherein the polar aprotic solvent comprises one or more of dimethylformamide, dimethylacetamide, dimethylsulfoxide, and N-methylpyrrolidone.

12. The process as claimed in claim 6, wherein the isolation comprises one or more of filtration, filtration under vacuum, evaporation, decantation, centrifugation, drying, and drying under vacuum.

13. The process as claimed in claim 6, further comprising cooling before isolating.

14. A process for the preparation of substantially pure febuxostat, the process comprising:
(a) obtaining a solution or a suspension of febuxostat in water having alkaline pH;
(b) adding one or more ester solvents;
(c) acidifying with acid;
(d) heating reaction mixture;
(e) separating organic phase; and
(f) isolating the substantially pure febuxostat.

15. The process as claimed in claim 14, wherein the ester comprises one or more of ethyl acetate, n-propyl acetate, isopropyl acetate, n-butyl acetate, and tertbutyl acetate.

16. The process as claimed in claim 14, wherein the acid comprises one or more of acetic acid, formic acid, hydrochloric acid, sulfuric acid, and phosphoric acid.

17. The process as claimed in claim 14, wherein the reaction mixture is heated from about ambient temperature to about reflux temperature.

18. The process as claimed in claim 14, wherein the isolation comprises one or more of distillation, distillation under vacuum, filtration, filtration under vacuum, evaporation, decantation, and centrifugation.

19. The process as claimed in claim 14, further comprising cooling before isolating the substantially pure febuxostat.

20. The process as claimed in claim 14, further comprising additional drying of the product obtained.

21. The process as claimed in claim 14, further comprising additional purifying the febuxostat obtained from a suitable organic solvent.

22. The process as claimed in claim 21, wherein the suitable organic solvent comprises one or more of methanol, ethanol, n-propanol, isopropanol, n-butanol, tert-amyl alcohol; aliphatic ketones like acetone, methylethylketone, methylisobutyl ketone; esters like ethyl acetate, n-butyl acetate, tert-butyl acetate, acetonitrile, dimethylformamide, dimethylacetamide, N-methylpyrrolidone, cyclohexane, n-heptane, n-hexane, methylene dichloride, and the like, and mixture thereof with water.

23. Febuxostat substantially free from dimer impurities of Formula (7b), (6b) and (1b).

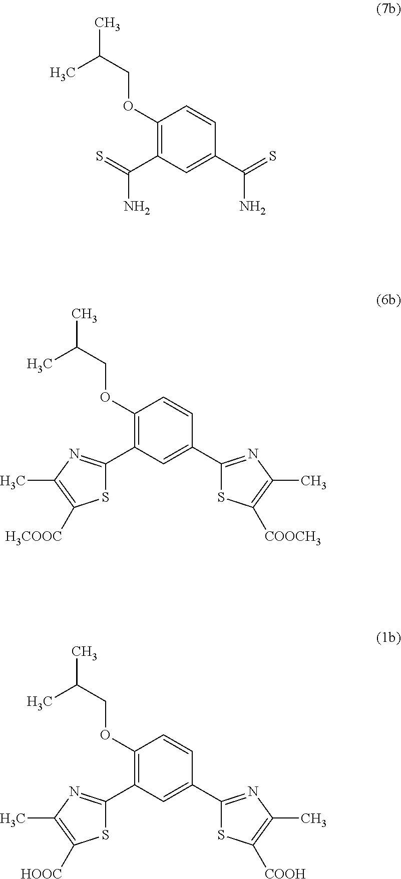

24. Substantially pure salts of febuxostat of Formula (IA), 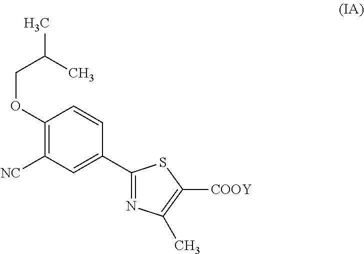
wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms, which are substantially free from dimer impurities of Formula (7b), (6b) and (1b).

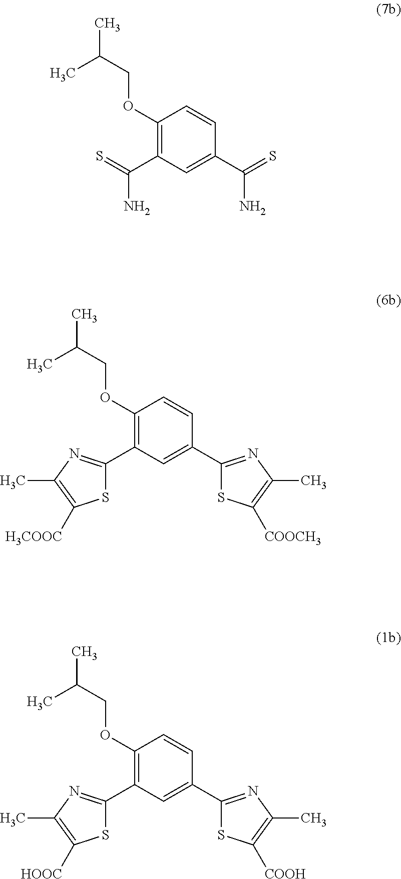

25. A process for the preparation of substantially pure febuxostat of Formula (I) 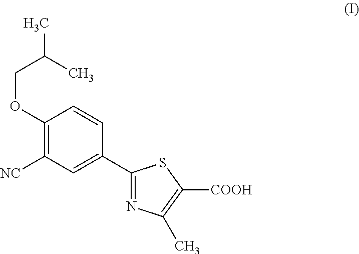 [SMILES: Cc1nc(-c2ccc(OCC(C)C)c(C#N)c2)sc1C(=O)O]
the process comprising:
(a) treating febuxostat with a suitable base to obtain febuxostat salt of Formula (IA) 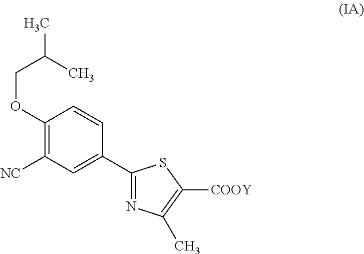
wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms;
(b) treating the febuxostat salt of Formula (IA) with an acid; and
(c) isolating the substantially pure febuxostat of Formula (I).

26. The process as claimed in claim 25, wherein the base is one or more of sodium hydroxide, potassium hydroxide, lithium hydroxide, calcium hydroxide, barium hydroxide, strontium hydroxide, zinc hydroxide, choline hydroxide, epolamine hydroxide, sodium carbonate, potassium carbonate, lithium carbonate, magnesium carbonate, calcium carbonate, sodium bicarbonate, potassium bicarbonate, lithium bicarbonate, sodium methoxide, potassium t-butoxide, magnesium methoxide, and the like.

27. The process as claimed in claim 25, wherein the acid is one or more of acetic acid, formic acid, hydrochloric acid, sulfuric acid, phosphoric acid, and the like.

28. Use of the compound of Formula (IA) in the process of manufacturing of 2-(3-cyano-4-isobutoxyphenyl)-4-methylthiazole-5-carboxylic acid.

29. A pharmaceutical composition comprising febuxostat salt of Formula (IA), 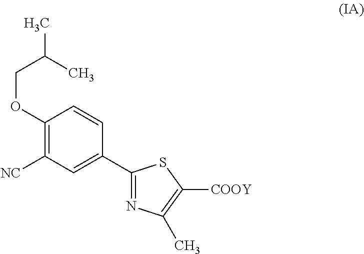
wherein Y is Na⁺, K⁺, Li⁺, Mg²⁺, Ca²⁺, Zn²⁺, Ba²⁺, Sr²⁺, choline, epolamine and N⁺(R)₄,
wherein R is alkyl with 1-4 carbon atoms, and one or more pharmaceutically acceptable carriers, excipients or diluents.
